(19) United States
(12) Plant Patent Application Publication
(10) Pub. No.: US 2009/0193552 P1
(43) Pub. Date: Jul. 30, 2009

(54) CHESTNUT PLANT NAMED 'AU BUCK III'

(50) ;

(75) Inventors: W. Alfred Dozier, JR., Opelika, AL (US); J.D. Norton, (US); Curtis J. Hansen, Opelika, AL (US)

Correspondence Address: HAVERSTOCK & OWENS LLP, 162 N WOLFE ROAD, SUNNYVALE, CA, 94086, US

(73) Assignee: Auburn University, an Alabama Corporation

(21) Appl. No.: 12/012,110

(22) Filed: Jan. 30, 2008

(51) Int. Cl. A01H 5/00

(52) U.S. Cl. PLT/152

(57) ABSTRACT

‘AU Buck III’ is a new and distinct Chinese chestnut cultivar that is blight resistant, precocious and an annual bearer. It produces a medium to heavy crop load each year and a medium sized nut. Nut drop begins about September 25 and continues for a 4-5 week period. A large percentage of the nuts drop during the first three weeks of the period that nuts drop from this cultivar. ‘AU Buck III’ is a small to medium sized tree that is round in shape with low, dense, and spreading branches that was 6.71 meters tall, with a canopy covering an area of 41.85 square meters at 20 years old. ‘AU Buck III’ is the third cultivar to mature and drop nuts in a series of four Chinese chestnut cultivars that will provide for a continuous nut drop of a high energy wildlife food source from late August through mid-to-late November.

### LATIN NAME OF THE GENUS AND SPECIES OF THE PLANT CLAIMED

Castanea P. Mill., Castanea mollissima Blume.

### VARIETY DENOMINATION

‘AU BUCK III’

### BACKGROUND OF THE INVENTION

A Chinese chestnut planting was established at Auburn University, Auburn, Ala., from nuts collected in China. The planting was established on the United States Department of Agriculture Horticulture Farm which in later years became the Mainstation Horticulture Farm. Precocious and prolific-bearing, blight resistant seedlings were selected for nut appearance, size and quality. Each generation of seedlings were the product of controlled mass pollination from the most promising seedlings selected from the previous generation. ‘AU Cropper’, ‘AU Leader’ and ‘AU Homestead’ were released from a second generation of approximately 2000 seedlings. A planting of third generation seedlings from controlled mass pollination of ‘AU Leader’, ‘AU Homestead’ and ‘AU Cropper’ was established at the Auburn University Piedmont Substation at Camp Hill, Ala.

### SUMMARY OF THE INVENTION

‘AU Buck III’ is from a sister line to ‘AU Leader,’ ‘AU Homestead’ and ‘AU Cropper’.

The present invention relates to a new and distinct Chinese chestnut cultivar that is blight resistant, precocious, produces a heavy crop annually, average nut size is medium (10.9 g), and begins nut drop about September 25 and continues for a 4-5 week period. A high percentage of the nuts produced drop during the first three weeks of the period that nuts drop from this cultivar. Nut drop from ‘AU Buck III’ begins about 2 weeks after nut drop from ‘AU Buck II’ begins. The medium sized nut is an excellent food source for wildlife such as deer and squirrels. The 20-year old tree of ‘AU Buck III’ is a small to medium sized tree in height that is 6.71 meters tall with a canopy of 7.29 meters wide and covers a canopy area of 41.85 square meters. The ‘AU Buck III’ tree has produced a heavy crop load each season for tree size and produced 20.3 kg (44.8 lbs) in 2006.

‘AU Buck III’ is the third cultivar to mature and drop nuts in a series of four Chinese chestnut cultivars that will provide for a continues nut drop of a high energy wildlife food source from late August through mid-to-late November. ‘AU Buck I’, ‘AU Buck II’ and ‘AU Buck IV’ are the other three cultivars, which are disclosed in U.S. patent application Ser. No. ______, filed on ______, and entitled “CHESTNUT PLANT NAMED ‘AU BUCK I’” [Attorney Docket No. AUB-07100], U.S. patent application Ser. No. ______, filed on ______, and entitled “CHESTNUT PLANT NAMED ‘AU BUCK II’” [Attorney Docket No. AUB-07200] and U.S. patent application Ser. No. ______, filed on ______, and entitled “CHESTNUT PLANT NAMED ‘AU BUCK IV’” [Attorney Docket No. AUB-07400], which are hereby incorporated by reference.

The new cultivar is able to be asexually reproduced by budding and grafting onto a seedling Chinese chestnut rootstock. The unique characteristics come true to form and are established and transmitted through asexual propagation.

### BRIEF DESCRIPTION OF THE DRAWINGS

FIG. 1 is a photograph of nuts of the ‘AU BUCK III’ cultivar.

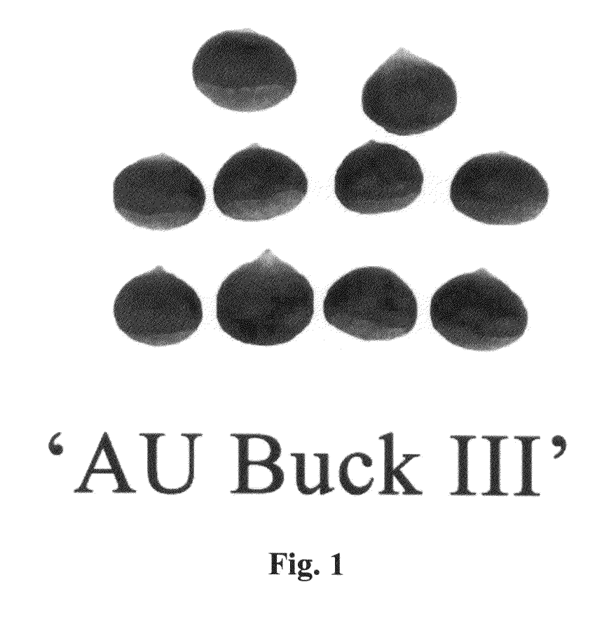

FIG. 2 is a photograph of nuts of the ‘AU BUCK III’ cultivar.

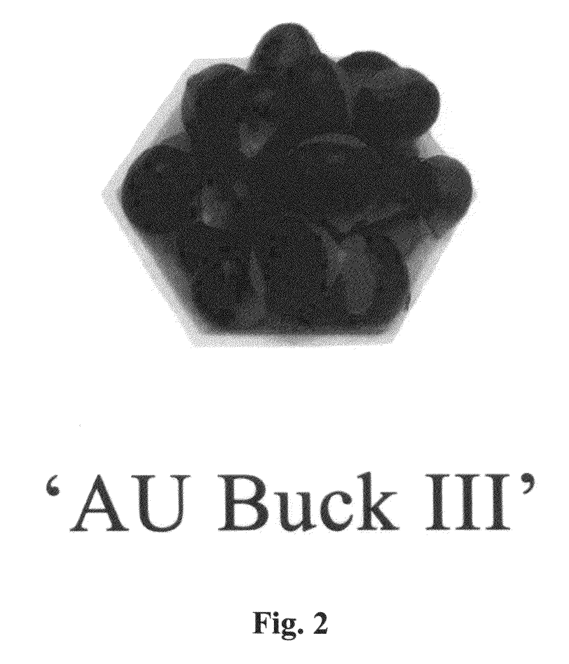

FIG. 3 is a photograph of a tree of the ‘AU BUCK III’ cultivar.

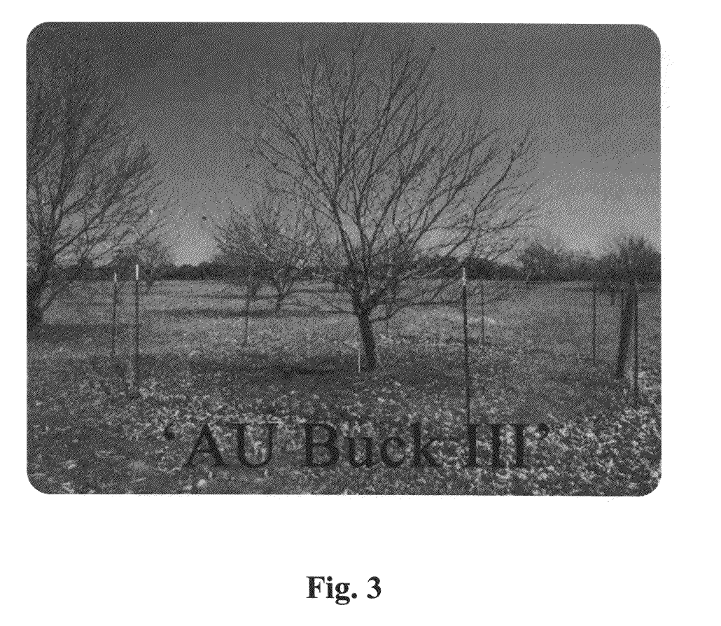

### DETAILED BOTANICAL DESCRIPTION

The Chinese chestnut, Castanea mollissima Blume, is a cold hardy temperate zone species native to China. It can be grown between 30° and 50° latitudes. The Chinese chestnut is resistant to chestnut blight fungus Cryphonectria parasitica. Generally, Chinese chestnuts are grown on a wide range of soils, but well-drained, deep and fertile soils are considered the best. Soils should be slightly acidic with pH 5.6-6.5. The name mollissima means soft hair and this species is recognized by dense hair on young leaves and downy yellow terminal parts of the shoots in winter. The leaf blade is thicker, and, in general, mature leaves are broader than those of other species. The nuts have a small scar or hilum. The pellicle or thin membranous skin on the nuts is thin and peels readily from the kernel. The trees are a spreading type and long-lived with a round top. The trees have bark with furrows and buds with 3-4 scales and leaves are 2 ranked, serrated with numerous parallel veins.

Chestnuts are monoecious and staminate flowers appear on erect cylindrical catkins with 10-20 stamens and 6-parted calyx. Pistillate flowers are borne on lower part of the upper staminate catkins and rarely on separate catkins usually 3 in a prickly symmetrical involucre with 7-9 styles and a 6-celled ovary. Nuts are small, brown with a pale scar at the base. Generally, 1-3 nuts per involucre or bur are present.

‘AU Buck III’ is a small to medium sized tree that is round in shape with low, dense, and spreading branches. The 20-year old tree is 6.71 meters (22.0 feet) tall, with a trunk diameter at breast height of 22.6 cm (8.90 inches). The canopy is 7.29 meters (23.90 feet) wide and covers an area of 41.85 square meters (450.48 square feet). The average nut weight is 10.9 g, average nut length is 28.4 mm (1.12 inches) and the average nut width is 30.4 cm (1.30 inches). The nuts start dropping about September 25 and continue to drop for a 4 to 5 week period. A high percent of the nuts produced drop during the first three weeks of the period that nuts drop from this cultivar. Nut drop from ‘AU Buck III’ begins about 2 weeks after nut drop from ‘AU Buck II’ begins. ‘AU Buck III’ is prolific and fruits annually.

In the planting at the Piedmont Substation, Camp Hill, Ala., accurate yields could not be obtained due to extremely heavy wildlife (deer and turkey) feeding. Therefore, the trees were rated for crop load each year. ‘AU Buck III’ has been a producer of a medium to large crop load each season. In the fall of 2006, individual trees were caged with 6 foot (1.83 meters) tall chicken wire prior to nut drop to exclude wildlife and nuts were picked up daily from the beginning through completion of nut drop. The 20-year old ‘AU Buck III’ tree produced 20.3 kg (44.8 pounds) of nuts in 2006. This yield is similar to reported yields of similar aged trees.

The table below illustrates the specific differences between the ‘AU BUCK III’ cultivar and the ‘REVIVAL’ cultivar.

The botanical details of this new and distinctive variety of
chestnut tree-with color definitions (except those in
common color terms) referenced to Royal Horticultural
Society's Colour Chart (RHS) and color was also determined
using an electronic spectrophotometer to determine hue angle
and Chroma (spectrophotometer model CM-2002; Minolta
Camera Co., Japan).
‘AU BUCK III’ CHESTNUT

Tree:
Size (at maturity)-small-twenty year old original tree is 6.71 meters tall
with a canopy width of 7.29 meters and a canopy area of 41.85 sq. meters
Tree shape is round.
Vigor-very vigorous
Trunk:
Form-upright with branches low and dense
Texture-relatively smooth
Color of bark-brown RHS N200A, Chroma C* 11.29, hue angle 80.74
Branches:
Form-spreading
Texture-relatively smooth
Lenticels-few, small
Branching habit-low and dense
Color-new wood: brown, RHS 200C, Chroma C* 16.06,
hue angle 68.53, mature wood: grey-brown RHS N199A
Chroma C* 13.18, hue angle 88.29
Foliage:
Quantity-abundant
Density-dense
Leaves:
Size-large. Length (cm) 19.3 (16.1-22.1) [20]
width (cm) 7.7 (5.0-9.5) [20]
leaf ratio 2.6(2.2-3.4) [20]
Shape-oblong-elliptic
leaf tip-acute to occasionally acuminate
leaf base-cuneate to rounded; equal, rarely oblique
Thickness-thick. Leaf venation 1° pinnate: 2° ± parallel, strongly
prominent abaxially
Texture-strongly coriaceous
Margin-coarsely serrate; teeth spreading, ascending
Petiole-length 1.4 cm (0.9-2.1) [20]
Petiole pubescence-mixed stellate and simple hairs
Color-adaxial surface, glabrous blade; simple and stellate hairs on main
veins medium to dark green; very shiny, RHS 147A
Chroma C* 16.23, hue angle 117.35
abaxial surface, densely stellate hairs mixed with occasional long straight
hairs on blade, long simple hairs on main veins, RHS 147B,
Chroma C* 21.68, hue angle 103.78
Bloom:
Amount of bloom-heavy
Color-anthesis, 161D greyed-yellow group, 157D green-white group,
155C white group
Blooming period-mid-May. After foliation in April.
Age at which tree starts flowering-early, 2-3 years alter graft
replacement.
Male flower-Catkin length (cm)-14.0 (9.6-19.8) [10]
Male flower-stamen number per catkin-10.8 (8.13) [10]
Female flower-flower number per bur-3
Female flower-style number per flower 7.7 (7.8) [3]
Crop:
Bearing-regular annual bearer
Productivity-prolific
Ripening period-long, nuts begin dropping about September 25
and continues for a 4-5 week period.
Distribution of nuts on tree-well distributed, fruits on terminals with
2-4 burs per terminal.
Tenacity-burs crack while on tree and nuts release and drop from bur.
Hull:
Description-spiny round bur
Size-2.34-2.56″ in diameter
Number of nuts-2-3 per bur
Dehiscence-splits easily when still on tree
Color-yellow-green at dehiscence, RHS N144C
Nut:
Size-medium; average size- 1.12″ × 1.30″ × 0.71″.
Average weight-10.9 grams 42 (36-46) nuts per pound.
Form-flattened on 1 side, occ. 2, hemispheric on the other side,
Blossom end-small obtuse tip
Basal end-broadly rounded
Color-brown, lustrous RHS 200B, Chroma C* 11.58, hue angle 36.17
Pubescence-mixed long and short, fine, white hairs densely covering
¼ of the distal end, otherwise very sparse to
glabrous elsewhere Shell-thin
Hardness of shell-relatively hard, yet not rigid
Texture of shell-smooth
Percentage of kernel to nut-very high-90-95%
Kernel:
Size-almost as large as nut size
Form-same as nut shape
Pellicle-thin
Flavor-excellent, very sweet
Color-straw color, RHS 152D, Chroma C* 48.19, hue angle 81.90
Resistance to insects: no unusual susceptibilities noted
Resistance to disease: no susceptibilities to disease noted
The chestnut tree and its nuts herein described may
vary in slight detail due to climatic and soil conditions
under which the variety may be grown; the present
description being of the variety as grown in Camp Hill,
Ala.

The botanical details of this variety of
chestnut tree-with color definitions (except those in
common color terms) referenced to Maerz and Pauxl
Dictionary of Color-are as follows:
‘REVIVAL’

Tree:
Size (at maturity)-large
Vigor-very vigorous
Trunk:
Form-upright with branches spreading in upper
reaches of tree.
Texture-relatively smooth
Color of bark-Silvergray (13-A-1)
Branches:
Form-strong
Texture-relatively smooth
Lenticels-few, small
Branching habit-spreading in upper region of tree
Color-new wood: reddish brown and glossy, mature
wood: silver gray
Foliage:
Quantity-abundant
Density-dense
Leaves:
Size-large. Average length-5-7″ (including petiole).
Average width -2″
Shape-oblong with acute tip and rounded base
Thickness-thick
Texture-smooth
Margin-dentate
Petiole-length: medium. Thickness: medium.
Color-Top side-glossy dark green (22-L.12).
Under side-lighter green (21-D-7).
Bloom:
Amount of bloom-heavy
Color-cream white (17-B-1)
Blooming period-late. After leaf out in April
Age at which tree starts flowering-early; 2-3 years
years after graft replacement.
Crop:
Bearing-regular (yearly) bearer
Productivity-prolific
Ripening period-short. September 15-October 1.
Distribution of nuts on tree-well distributed
Tenacity-burrs crack while on tree and nuts easily
release, many falling by themselves
Hull:
Description-spiny, round burr
Size-3-4″ in diameter
Number of nuts-2-3 per burr
Dehiscence-splits easily when still on tree. Some
entire burrs split and fall to ground
Color-brown (15-A-8)
Nut:
Size-large. Average size-1⅛″ X1⅛″ X1″ thick.
Average weight-24-32 nuts per pound
Form-broad and ovoid on one side, flat on other side
Blossom end-pointed tip
Basal end-flattened
Color-India Red (7-L-6).
Shell-thin
Hardness of shell-relatively hard, yet not rigid
Texture of shell-smooth
Percentage of kernel to nut-very high (95%)
Kernel:
Size-almost as large as nut size
Form-same as nut shape
Pellicle-thin
Flavor-excellent. Very sweet.
Color-Oyster white (10-B-1)
Resistance to insects: no unusual susceptibilities noted
Resistance to disease: very high inherent resistance to
chestnut bark fungus (Endothia parastica), no
other susceptibilities to any other disease
The chestnut tree and its nuts herein described may
vary in slight detail due to climatic and soil conditions
under which the variety may be grown; the present
description being of the variety as grown in Alachua,
Fla.

What is claimed is:

1. A new and distinct cultivar of the species Castanea mollissima Blume named ‘AU BUCK III’ as described and illustrated herein.
